(19) United States
(12) Patent Application Publication
(10) Pub. No.: US 2019/0298876 A1
(43) Pub. Date: Oct. 3, 2019

(54) Air Freshener Art and Non-Marring Attachment

(71) Applicant: Charlene F. Chessum, Stockton, CA (US)

(72) Inventor: Charlene F. Chessum, Stockton, CA (US)

(21) Appl. No.: 15/496,097

(22) Filed: Apr. 25, 2017

Related U.S. Application Data
(60) Provisional application No. 62/391,474, filed on May 2, 2016.

(51) Int. Cl. A61L 9/12

(52) U.S. Cl. CPC A61L 9/12; A61L 2209/15

(57) ABSTRACT

The present invention relates generally to personal air fresheners that a person would utilize in their personal space to freshen the air in the vehicle, cubicle, office, bathroom, bedroom, dorm room, apartment or any other personal space where one would use an air freshener. Even more particularly, this invention relates to such air fresheners that is decorative and can be moved from one location to another in an artful manner without marring the surface that the air freshener is attached to. This invention is designed to allow the user to be creative in displaying their air freshener in an artful way.

### BACKGROUND

Air fresheners of various designs have been used by a person to freshen the air in the immediate and surrounding area. As well known, a typical air freshener generally comprises a housing to hold the liquid or solid, cardboard that is cut into shapes and has a scent embedded in the cardboard, a clay type substance that has a scent embedded, a spray that contains a concentrated scent or lighter scent depending on the use, a housing that is operated by electricity that uses essential oils to freshen the air, scented candles, wax melts, scented beads, sachets containing various scented beads or natural items such as pine, terra cotta or clay shapes with scents embedded, automatic air fresheners, clay and powder that can be used to sprinkle on the carpet.

Air fresheners have become more decorative over the years, yet still not very versatile. Air fresheners still must be contained in a housing of some sort and then hung by a string, sat on a solid surface, laid on a surface, clipped to a surface, stuck to a surface, plugged in or sprayed into the air.

As generally well known by persons who utilize an air freshener they are more utilitarian that attractive and versatile. Unfortunately, this results in air freshens being placed in less than ideal locations, limits on where you can locate an air freshener and limits on the attractiveness of the air freshener.

The problem with limited locations is the air fresher is not located in the ideal location for creating a pleasant environment, or a pleasing visual experience. The Air Freshener Art and Non-Marring Attachment will elevate these situations. Example: An air freshener in the shape of a flower can have a solid center and the petals be made of a porous material that will absorb the desired scent and slowly release it into the air or gel filled petals that slowly release the desired scent. The air freshener could be located behind the flower and emit the desired scent. An air freshener in the shape of a flower could have a center that contains the air freshener as a solid, gel, porous material containing the desired scent and slowly release the scent and the petals be made of silk, plastic, rubber, silicone, paper or some other surface that one would find pleasing. Example: An air freshener in the shape of a flower could allow a snap in component so the user could choose their petals and their center and put them together creating their own unique individual piece and scent combination. Example: An air freshener in the shape of a butterfly can have a solid body and the wings be made of a porous material that will absorb the desired scent and slowly release it into the air and the wings be made of silk, plastic, rubber, silicone, paper or some other surface that one would find pleasing. An air freshener in the shape of a butterfly could also have a solid body with wings that contain the desired scent. The desired scent could be in wings made of silk, rubber, silicone, paper, cardboard, or any surface that would absorb a desired scent and allow it to be released in a slow process. Example: An air freshener in the shape of a butterfly could allow a snap in component so the user could choose their wings and their body and put them together creating their own unique individual piece and scent combination.

Attempts have been made to create more attractive ways of utilizing and displaying air fresheners. For instance, U.S. Pat. No. 6,391,398B1 displays an air freshener in an artful way and is designed to slt on a surface. U.S. Patent No. 20110108632A1 attaches an air freshener to a vent using a clip. U.S. Pat. No. 7,028,917B2 which allows the device in which the air freshener is placed to hang from an object. U.S. Pat. No. 7,159,792B2 includes a scent material carried by and dispersible through a coherent, flexible polymer carrier material—the pad can be selectively re-secured at a different position on the surface. U.S. Pat. No. D323554 has a scented material carried by a housing unit that is attached to a surface. The housing unit is then twisted to various positions allowing the amount of scent you would like. The housing unit is attached using a small amount of adhesive tape that doesn't allow movement to another location.

Each of these patents cover a different area that helps individuals solve the decorative air freshener issue; however, none of them do it in such a way that is both artful, decorative and easily moved from one location to another in a non-marring fashion. The air freshener art and non-marring attachment allows an individual to choose an artistically designed housing with a desirable scent embedded in the housing in a variety of ways. It allows the air freshener to be moved from one location to another location without marring the surface by using a non-marring removable glue type substance as the backing allowing you to use it on any location such as mirror, dashboards, cubicles, walls, or anywhere you can think of, a clip that allows you to use it on a vent, visor, seatbelt, backpack, chair, table—anywhere you would want to clip a decorative air freshener, a magnet that allows you to use it on a locker, file cabinet, refrigerator, stove, BBQ, anything that a magnet would adhere to.

### SUMMARY

The air freshener allows for various designs to be created as a whole or as parts that one can create their own look and collection of scents creating a unique scent. The air freshener allows for use in various locations without marring the surface allowing the user a variety of ways to use and display the air freshener in an artful manner.

### DESCRIPTION OF THE DRAWINGS

FIG. 1 shows the butterfly as a whole.

FIG. 2 shows the flower as a whole.

FIG. 3 show the butterfly and its various parts.

FIG. 4 shows that cloth or fabric can be used for both the outer wings comprised of 1, 6, 8 and 13 shown in FIG. 3 and the inner wings comprised 4, 5, 10, and 12 shown in FIG. 3. Any of these sections can have the scent embedded or have a solid scent housing. The body comprised of 2, 3, 7, 9 and 11 shown in FIG. 3 can contain the scent using a solid, a gel, spray, liquid or scent absorbing material as shown in FIG. 21 or no scent at all. 1, 3, 4, 5, 6, 8, 9, 10, 11, 12, and 13 as shown in FIG. 3 can all contain scents and/or any variety of parts can contain scents or no scent at all. The scent can be housed behind or in front of the decorative item as a whole. Or any combination can be used.

FIG. 5 shows that cloth or fabric can be used for the outer wings comprised of 1, 6, 8 and 13 shown in FIG. 3 and synthetic resin or plastic can be used for the inner wings comprised 4, 5, 10, and 12 shown in FIG. 3. Any of these sections can have the scent embedded or have a solid scent housing. The body comprised of 2, 3, 7, 9 and 11 shown in FIG. 3 can contain the scent using a solid, a gel, spray, liquid or scent absorbing material as shown in FIG. 21 or no scent at all. 1, 3, 4, 5, 6, 8, 0, 10, 11, 12, and 13 as shown in FIG. 3 can all contain scents and/or any variety of parts can contain scents or no scent at all. The scent can be housed behind or in front of the decorative item as a whole. Or any combination can be used.

FIG. 6 shows that cloth or fabric can be used for the outer wings comprised of 1, 6, 8 and 13 shown in FIG. 3 and paper can be used for the inner wings comprised 4, 5, 10, and 12 shown in FIG. 3. Any of these sections can have the scent embedded or have a solid scent housing. The body comprised of 2, 3, 7, 9 and 11 shown in FIG. 3 can contain the scent using a solid, a gel, spray, liquid or scent absorbing material as shown in FIG. 21 or no scent at all. 1, 3, 4, 5, 6, 8, 9, 10, 11, 12, and 13 as shown in FIG. 3 can all contain scents and/or any variety of parts can contain scents or no scent at all. The scent can be housed behind or in front of the decorative item as a whole. Or any combination can be used.

FIG. 7 shows that cloth or fabric can be used for the outer wings comprised of 1, 6, 8 and 13 shown in FIG. 3 and synthetic sponge can be used for the inner wings comprised 4, 5, 10, and 12 shown in FIG. 3. Any of these sections can have the scent embedded or have a solid scent housing. The body comprised of 2, 3, 7, 9 and 11 shown in FIG. 3 can contain the scent using a solid, a gel, spray, liquid or scent absorbing material as shown in FIG. 21 or no scent at all. 1, 3, 4, 5, 6, 8, 9, 10, 11, 12, and 13 as shown in FIG. 3 can all contain scents and/or any variety of parts can contain scents or no scent at all. The scent can be housed behind or in front of the decorative item as a whole. Or any combination can be used.

FIG. 8 shows that paper can be used for the outer wings comprised of 1, 6, 8 and 13 shown in FIG. 3 and cloth or fabric can be used for the inner wings comprised 4, 5, 10, and 12 shown in FIG. 3. Any of these sections can have the scent embedded or have a solid scent housing. The body comprised of 2, 3, 7, 9 and 11 shown in FIG. 3 can contain the scent using a solid, a gel, spray, liquid or scent absorbing material as shown in FIG. 21 or no scent at all. 1, 3, 4, 5, 6, 8, 9, 10, 11, 12, and 13 as shown in FIG. 3 can all contain scents and/or any variety of parts can contain scents or no scent at all. The scent can be housed behind or in front of the decorative item as a whole. Or any combination can be used.

FIG. 9 shows that paper can be used for the outer wings comprised of 1, 6, 8 and 13 shown in FIG. 3 and paper can be used for the inner wings comprised 4, 5, 10, and 12 shown in FIG. 3. Any of these sections can have the scent embedded or have a solid scent housing. The body comprised of 2, 3, 7, 9 and 11 shown in FIG. 3 can contain the scent using a solid, a gel, spray, liquid or scent absorbing material as shown in FIG. 21 or no scent at all. 1, 3, 4, 5, 6, 8, 9, 10, 11, 12, and 13 as shown in FIG. 3 can all contain scents and/or any variety of parts can contain scents or no scent at all. The scent can be housed behind or in front of the decorative item as a whole. Or any combination can be used.

FIG. 10 shows that paper can be used for the outer wings comprised of 1, 6, 8 and 13 shown in FIG. 3 and synthetic resin or plastic can be used for the inner wings comprised 4, 5, 10, and 12 shown in FIG. 3. Any of these sections can have the scent embedded or have a solid scent housing. The body comprised of 2, 3, 7, 9 and 11 shown in FIG. 3 can contain the scent using a solid, a gel, spray, liquid or scent absorbing material as shown in FIG. 21 or no scent at all. 1, 3, 4, 5, 6, 8, 9, 10, 11, 12, and 13 as shown in FIG. 3 can all contain scents and/or any variety of parts can contain scents or no scent at all. The scent can be housed behind or in front of the decorative item as a whole. Or any combination can be used.

FIG. 11 shows that paper can be used for the outer wings comprised of 1, 6, 8 and 13 shown in FIG. 3 and synthetic sponge can be used for the inner wings comprised 4, 5, 10, and 12 shown in FIG. 3. Any of these sections can have the scent embedded or have a solid scent housing. The body comprised of 2, 3, 7, 9 and 11 shown in FIG. 3 can contain the scent using a solid, a gel, spray, liquid or scent absorbing material as shown in FIG. 21 or no scent at all. 1, 3, 4, 5, 6, 8, 9, 10, 11, 12, and 13 as shown in FIG. 3 can all contain scents and/or any variety of parts can contain scents or no scent at all. The scent can be housed behind or in front of the decorative item as a whole. Or any combination can be used.

FIG. 12 shows that synthetic resin can be used for the outer wings comprised of 1, 6, 8 and 13 shown in FIG. 3 and cloth or fabric can be used for the inner wings comprised 4, 5, 10, and 12 shown in FIG. 3. Any of these sections can have the scent embedded or have a solid scent housing. The body comprised of 2, 3, 7, 9 and 11 shown in FIG. 3 can contain the scent using a solid, a gel, spray, liquid or scent absorbing material as shown in FIG. 21 or no scent at all. 1, 3, 4, 5, 6, 8, 9, 10, 11, 12, and 13 as shown in FIG. 3 can all contain scents and/or any variety of parts can contain scents or no scent at all. The scent can be housed behind or in front of the decorative item as a whole. Or any combination can be used.

FIG. 13 shows that synthetic resin can be used for the outer wings comprised of 1, 6, 8 and 13 shown in FIG. 3 and paper can be used for the inner wings comprised 4, 5, 10, and 12 shown in FIG. 3. Any of these sections can have the scent embedded or have a solid scent housing. The body comprised of 2, 3, 7, 9 and 11 shown in FIG. 3 can contain the scent using a solid, a gel, spray, liquid or scent absorbing material as shown in FIG. 21 or no scent at all. 1, 3, 4, 5, 6, 8, 9, 10, 11, 12, and 13 as shown in FIG. 3 can all contain scents and/or any variety of parts can contain scents or no scent at all. The scent can be housed behind or in front of the decorative item as a whole. Or any combination can be used.

FIG. 14 shows that synthetic resin or plastic can be used for the outer wings comprised of 1, 6, 8 and 13 shown in FIG. 3 and synthetic resin or plastic can be used for the inner wings comprised 4, 5, 10, and 12 shown in FIG. 3. Any of these sections can have the scent embedded or have a solid scent housing. The body comprised of 2, 3, 7, 9 and 11 shown in FIG. 3 can contain the scent using a solid, a gel, spray, liquid or scent absorbing material as shown in FIG. 21 or no scent at all. 1, 3, 4, 5, 6, 8, 9, 10, 11, 12, and 13 as shown in FIG. 3 can all contain scents and/or any variety of parts can contain scents or no scent at all. The scent can be housed behind or in front of the decorative item as a whole. Or any combination can be used.

FIG. 15 shows that synthetic resin or plastic can be used for the outer wings comprised of 1, 6, 8 and 13 shown in FIG. 3 and synthetic sponge can be used for the inner wings comprised 4, 5, 10, and 12 shown in FIG. 3. Any of these sections can have the scent embedded or have a solid scent housing. The body comprised of 2, 3, 7, 9 and 11 shown in FIG. 3 can contain the scent using a solid, a gel, spray, liquid or scent absorbing material as shown in FIG. 21 or no scent at all. 1, 3, 4, 5, 6, 8, 9, 10, 11, 12, and 13 as shown in FIG. 3 can all contain scents and/or any variety of parts can contain scents or no scent at all. The scent can be housed behind or in front of the decorative item as a whole. Or any combination can be used.

FIG. 16 shows that synthetic resin or plastic can be used for the outer wings comprised of 1, 6, 8 and 13 shown in FIG. 3 and cloth or fabric can be used for the inner wings comprised 4, 5, 10, and 12 shown in FIG. 3. Any of these sections can have the scent embedded or have a solid scent housing. The body comprised of 2, 3, 7, 9 and 11 shown in FIG. 3 can contain the scent using a solid, a gel, spray, liquid or scent absorbing material as shown in FIG. 21 or no scent at all. 1, 3, 4, 5, 6, 8, 9, 10, 11, 12, and 13 as shown in FIG. 3 can all contain scents and/or any variety of parts can contain scents or no scent at all. The scent can be housed behind or in front of the decorative item as a whole. Or any combination can be used.

FIG. 17 shows that synthetic sponge can be used for the outer wings comprised of 1, 6, 8 and 13 shown in FIG. 3 and paper can be used for the inner wings comprised 4, 5, 10, and 12 shown in FIG. 3. Any of these sections can have the scent embedded or have a solid scent housing. The body comprised of 2, 3, 7, 9 and 11 shown in FIG. 3 can contain the scent using a solid, a gel, spray, liquid or scent absorbing material as shown in FIG. 21 or no scent at all. 1, 3, 4, 5, 6, 8, 9, 10, 11, 12, and 13 as shown in FIG. 3 can all contain scents and/or any variety of parts can contain scents or no scent at all. The scent can be housed behind or in front of the decorative item as a whole. Or any combination can be used.

FIG. 18 shows that synthetic sponge can be used for the outer wings comprised of 1, 6, 8 and 13 shown in FIG. 3 and synthetic resin or plastic can be used for the inner wings comprised 4, 5, 10, and 12 shown in FIG. 3. Any of these sections can have the scent embedded or have a solid scent housing. The body comprised of 2, 3, 7, 9 and 11 shown in FIG. 3 can contain the scent using a solid, a gel, spray, liquid or scent absorbing material as shown in FIG. 21 or no scent at all. 1, 3, 4, 5, 6, 8, 9, 10, 11, 12, and 13 as shown in FIG. 3 can all contain scents and/or any variety of parts can contain scents or no scent at all. The scent can be housed behind or in front of the decorative item as a whole. Or any combination can be used.

FIG. 19 shows that synthetic sponge can be used for the outer wings comprised of 1, 6, 8 and 13 shown in FIG. 3 and synthetic sponge can be used for the inner wings comprised 4, 5, 10, and 12 shown in FIG. 3. Any of these sections can have the scent embedded or have a solid scent housing. The body comprised of 2, 3, 7, 9 and 11 shown in FIG. 3 can contain the scent using a solid, a gel, spray, liquid or scent absorbing material as shown in FIG. 21 or no scent at all. 1, 3, 4, 5, 6, 8, 9, 10, 11, 12, and 13 as shown in FIG. 3 can all contain scents and/or any variety of parts can contain scents or no scent at all. The scent can be housed behind or in front of the decorative item as a whole. Or any combination can be used.

FIG. 20 shows that synthetic sponge can be used for the outer wings comprised of 1, 6, 8 and 13 shown in FIG. 3 and cloth or fabric can be used for the inner wings comprised 4, 5, 10, and 12 shown in FIG. 3. Any of these sections can have the scent embedded or have a solid scent housing. The body comprised of 2, 3, 7, 9 and 11 shown in FIG. 3 can contain the scent using a solid, a gel, spray, liquid or scent absorbing material as shown in FIG. 21 or no scent at all. 1, 3, 4, 5, 6, 8, 9, 10, 11, 12, and 13 as shown in FIG. 3 can all contain scents and/or any variety of parts can contain scents or no scent at all. The scent can be housed behind or in front of the decorative item as a whole. Or any combination can be used.

FIG. 21 shows that synthetic sponge, cloth or fabric, paper or synthetic resin or plastic can be used for the bodies of the butterfly. Any of these sections can have the scent embedded or have a solid scent housing. Section 3, 9 and 11 can contain the scent using a solid, a gel, a liquid, spray or scent absorbing material and allows the non-marring substance to be applied to the underside.

FIG. 22 shows the flower as a whole and its parts.

FIG. 23 shows that cloth or fabric petals can be used. Any one of the sections 1, 2, 3, 4, 5, 6, 7, 8, 9, 10, 12, 13 and 14 can have a scent embedded, have a solid scent housing or the scent behind each petal, a single petal or combination of petals.

FIG. 24 shows that paper petals can be used. Any of these sections can have the scent embedded or have a solid scent housing. Any one of the sections 1, 2, 3, 4, 5, 6, 7, 8, 9, 10, 12, 13 and 14 can have a scent embedded, have a solid scent housing or the scent behind each petal, a single petal or combination of petals.

FIG. 25 shows that synthetic resin or plastic resin can be used. Any of these sections can have the scent embedded or have a solid scent housing. Any one of the sections 1, 2, 3, 4, 5, 6, 7, 8, 9, 10, 12, 13 and 14 can have a scent embedded, have a solid scent housing or the scent behind each petal, a single petal or combination of petals.

FIG. 26 shows that synthetic sponge petals can be used. Any of these sections can have the scent embedded or have a solid scent housing. Any one of the sections 1, 2, 3, 4, 5, 6, 7, 8, 9, 10, 12, 13 and 14 can have a scent embedded, have a solid scent housing or the scent behind each petal, a single petal or combination of petals.

FIG. 27 shows that paper, cloth or fabric, synthetic sponge, synthetic resin or plastic can be used for the center of the flower. The center can be made out of any one of these types of materials and have the scent embedded, behind it or can simply be a solid housing containing the scent, such as a gel scent. Any of the various materials shown in FIG. 27 can be added to any FIG. 23, 24, 25 or 26.

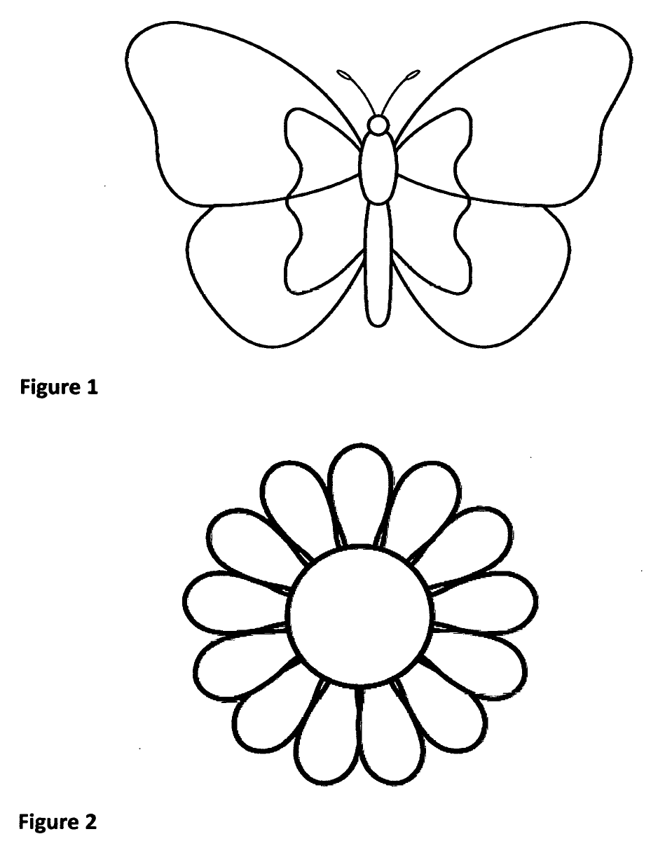

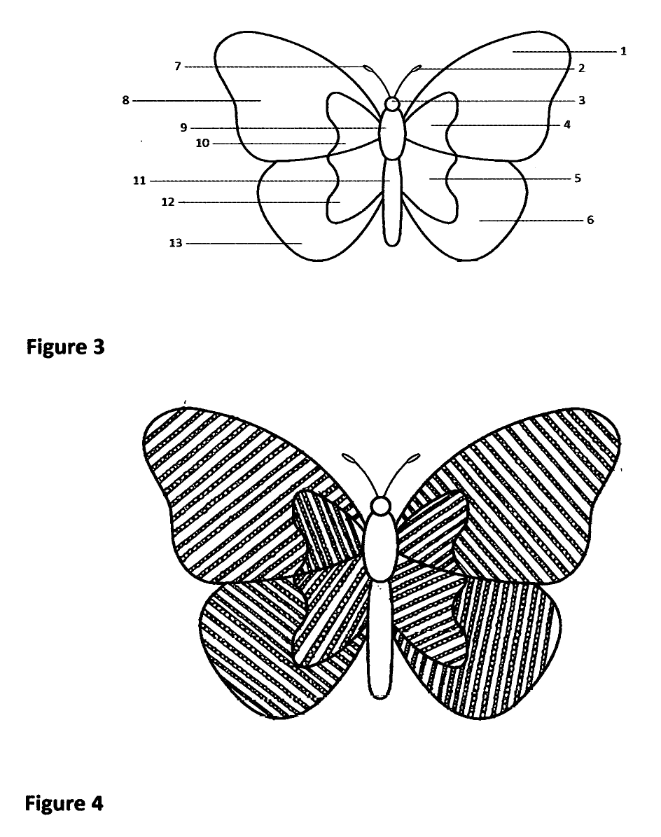

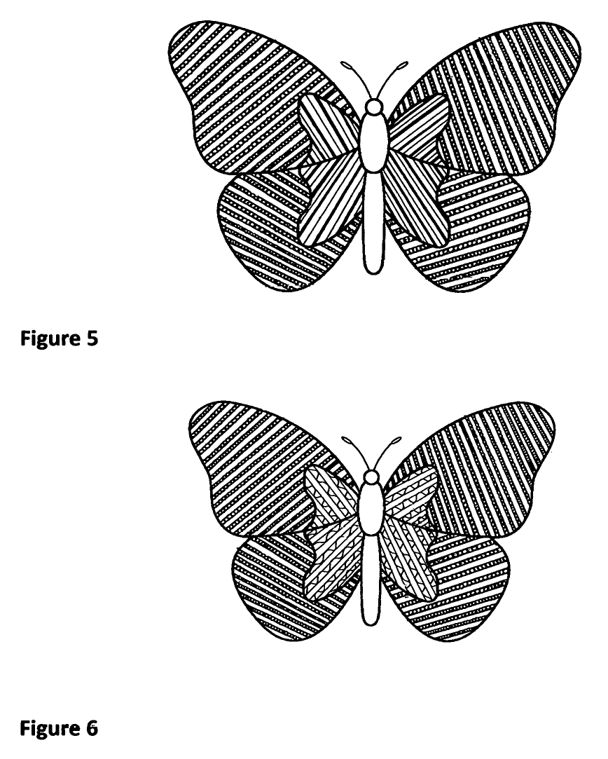

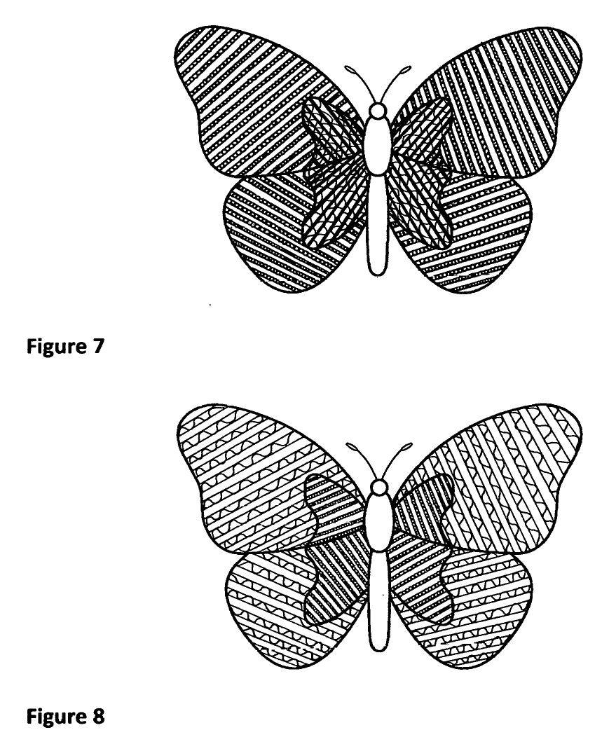

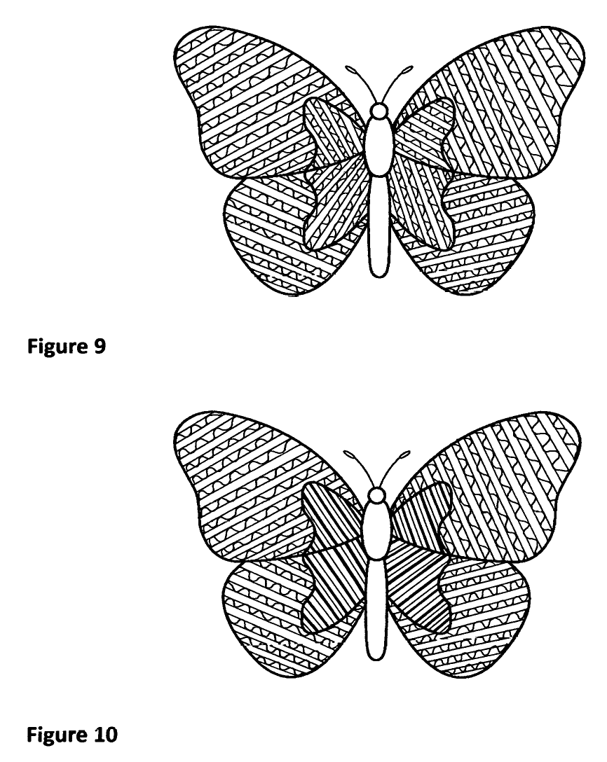

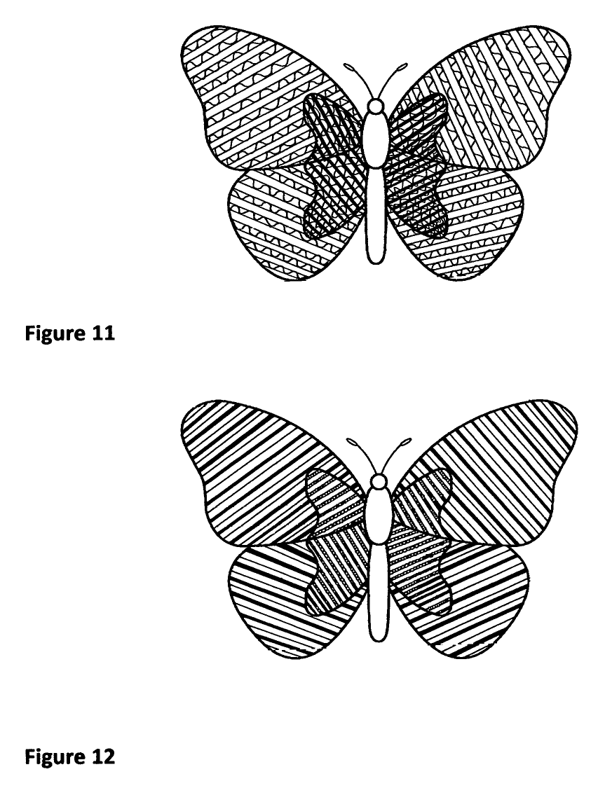

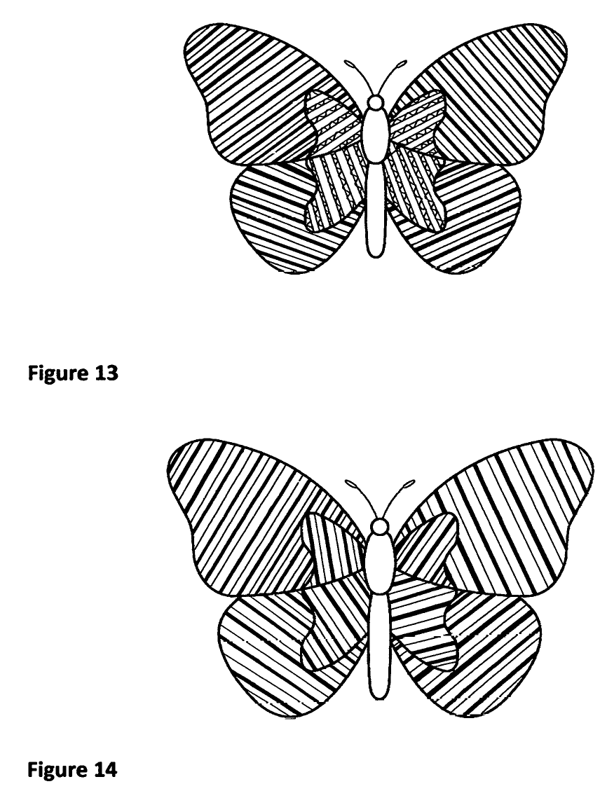

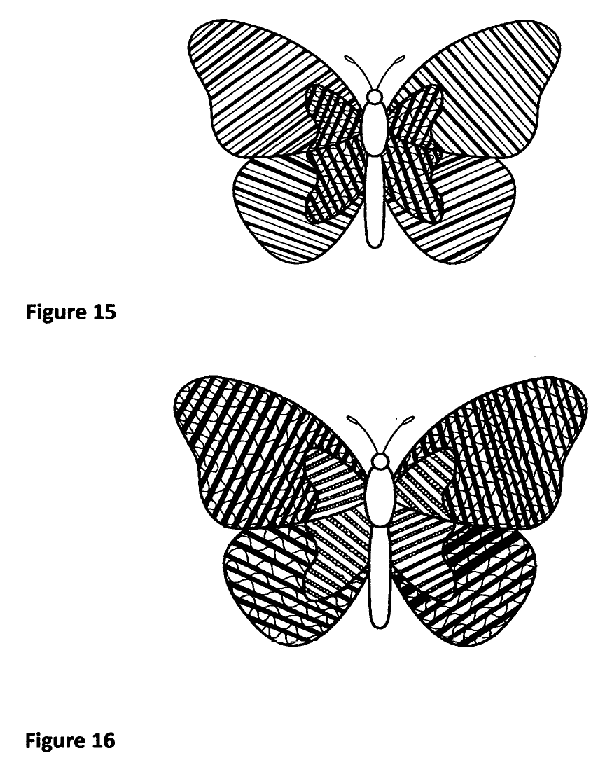

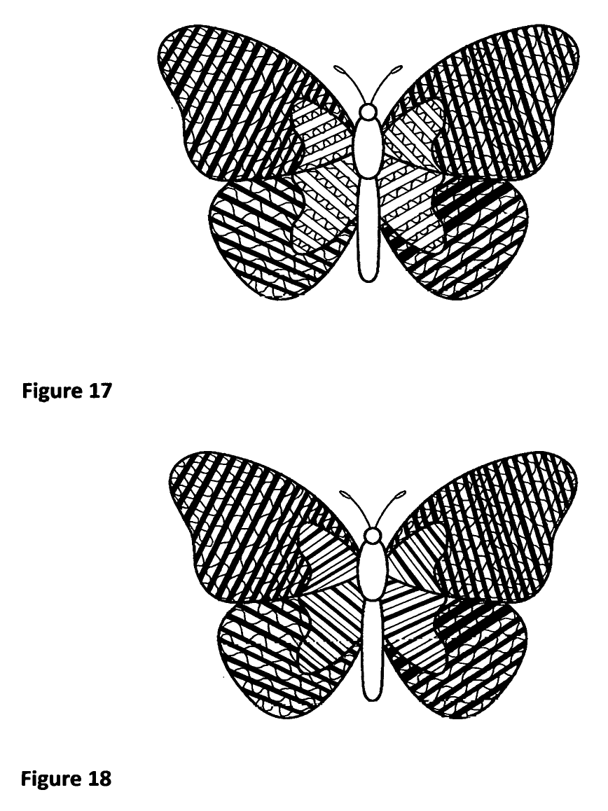

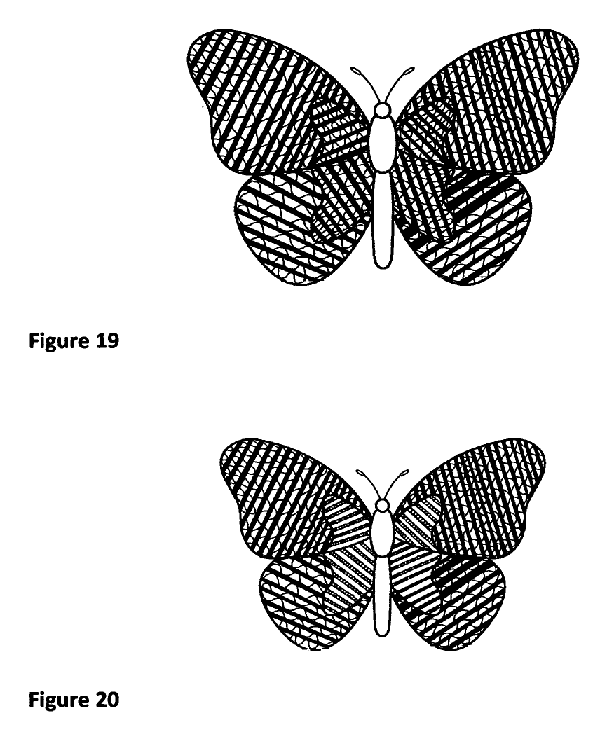

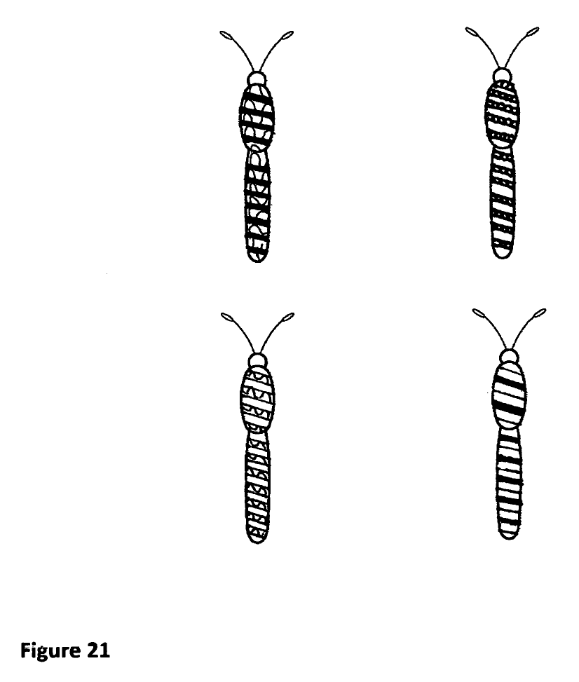

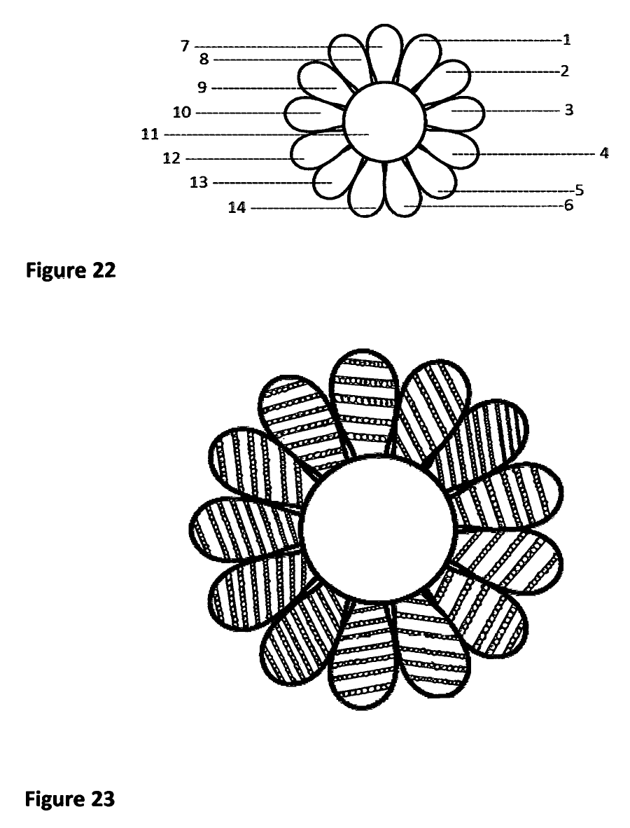

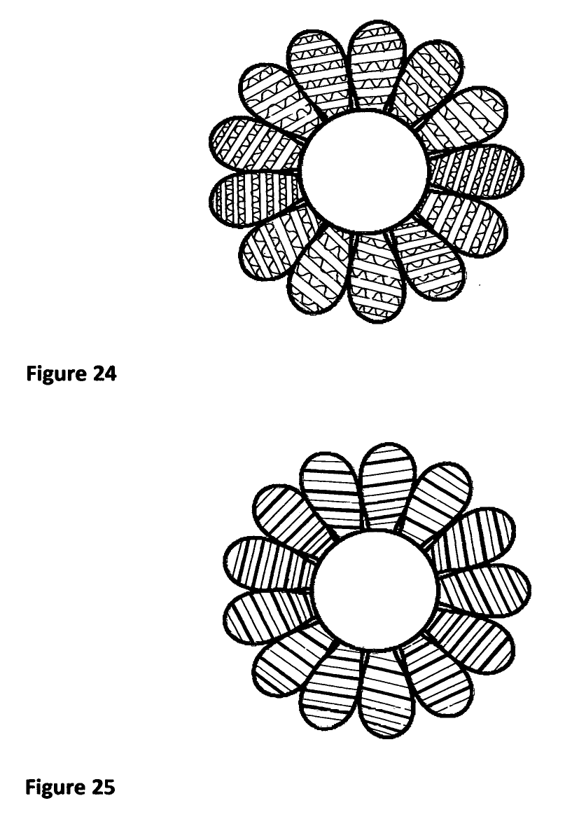

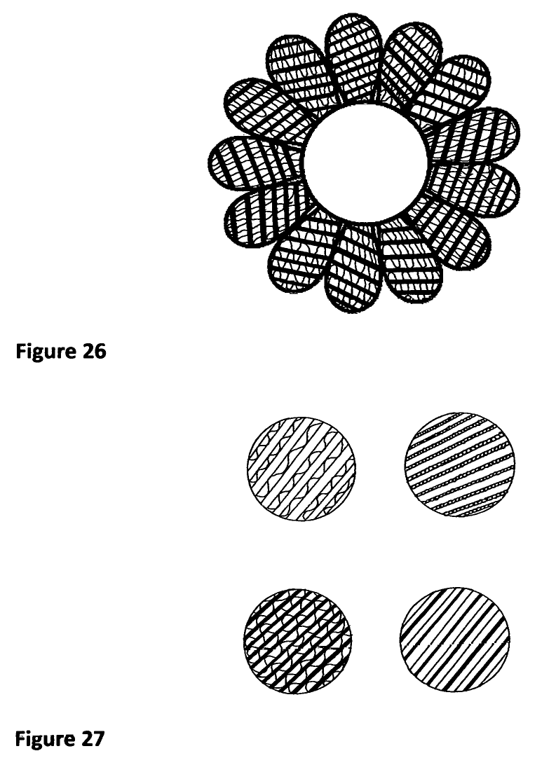

### DESCRIPTION OF THE INVENTION

The invention is any item of art that scent can be added to the item entirely, to parts of the art or to a housing that is attached to the art and that a non-marring substance can be applied to so that the air freshener art can be attached to the area and/or location that you would like.

1. A decorative item that is flat or 3D in nature and is small. The item can be made of anything that can absorb a desired scent and release the scent in a slow manner.

2. A portion of the decorative item that is flat or 3D in nature is small. The item can be made of anything that can absorb a desired scent and release the scent in a slow manner.

3. A decorate items that is flat or 3D in nature and is small. The item can be made of anything that is complimentary to the item made of a material that can absorb and release a desired scent in a slow manner.

4. The materials in claims 1, 2 and 3 can be used together in any combination of ways.

5. The air freshener, as a whole, can be moved from one location to another without marring the surface that is was attached to.

6. Mix and Match—The various items can be sold individually and put together in different ways allowing the user to create their “own” decorative piece with their desired scent.
